(19) United States
(12) Patent Application Publication
(10) Pub. No.: US 2014/0011686 A1
(43) Pub. Date: Jan. 9, 2014

(54) SELECTION OF SINGLE NUCLEIC ACIDS BASED ON OPTICAL SIGNATURE

(71) Applicant: PathoGenetix, Inc., Woburn, MA (US)

(72) Inventor: Rudolf Gilmanshin, Framingham, MA (US)

(73) Assignee: PathoGenetix, Inc., Woburn, MA (US)

(21) Appl. No.: 13/865,526

(22) Filed: Apr. 18, 2013

Related U.S. Application Data
(60) Provisional application No. 61/635,221, filed on Apr. 18, 2012.
(60) Provisional application No. 61/790,721, filed on Mar. 15, 2013.

(51) Int. Cl. C12Q 1/68

(52) U.S. Cl. CPC C12Q 1/689
USPC 506/2; 506/9; 506/39

(57) ABSTRACT

The invention provides, inter alia, methods for analyzing populations nucleic acids including nucleic acids from different subjects or different samples such as bacteria in biomes, and for identifying nucleic acids present in such populations. A non-limiting example is an analysis of bacteria in biomes. Another example is an analysis of DNA from different human subjects.

### RELATED APPLICATIONS

This application claims the benefit under 35 U.S.C. §119(e) of U.S. Provisional Application Ser. No. 61/635,221, filed on Apr. 18, 2012, and entitled SINGLE-MOLECULE SORTING AND MAPPING OF LONG DNA FRAGMENTS FOR SEQUENCING-BASED ANALYSIS, and U.S. Provisional Application Ser. No. 61/790,721 filed on Mar. 15, 2013, and entitled SELECTION OF SINGLE NUCLEIC ACIDS BASED ON OPTICAL SIGNATURE, the entire contents of both of which are incorporated by reference herein.

### FIELD OF INVENTION

The invention relates to the analysis of nucleic acids such as nucleic acids from populations of cells of differing sources, species and/or strains, including for example bacterial populations or human cells.

### BACKGROUND OF INVENTION

De novo sequencing is typically not required for the vast majority of applications, including for example some types of human diagnostics and pharmacogenomics. Instead, sequencing may be used to identify genetic differences between individuals, using a process called targeted re-sequencing. Similarly, analysis of a bacterial population does not need to include complete sequencing of the whole population. Instead, only parts of a bacterial genome that sufficiently identify the host genome can be selected for analysis.

### SUMMARY OF INVENTION

The methods of the invention can be used, inter alia, to isolate fragments of genomic DNA containing specific genes of interest, or manifesting specific probe hybridization profile or barcodes of interest, from multiple sources and from large genomes. The fragment sorting may be accompanied by simultaneous measurement of genomic maps that complement the information about the specific genes, profiles and/or barcodes and can help to build sequence contigs. This technology provides efficient and rapid analysis, selecting only a fraction of genomic material, relevant for an application or analysis. These methods can be used to more rapidly type and obtain sequence for nucleic acid fragments of interest.

The invention contemplates, in some aspects, the analysis of microbiomes (i.e., populations including multiple bacteria) based on sequencing their housekeeping genes, using the method and devices provided herein. The invention contemplates, in other aspects, the analysis of human genomes. In further aspects, the invention provides a device to physically select a limited amount of nucleic acid (e.g., DNA) for further analysis (e.g., targeted resequencing) from a bulk nucleic acid population such as for example genomic DNA including genomic DNA isolated from a microbiome sample. Various aspects and embodiments of the invention are described in terms of DNA, but it is to be understood that other nucleic acids may be used also and that the invention is not limited to analysis of DNA molecules only.

Thus, in one aspect, the invention provides a method comprising (1) labeling nucleic acid fragments with a nucleic acid probe that is a specific probe, (2) labeling the fragments with a non-specific probe, wherein the specific probe and the non-specific probe are labeled with different detectable labels, and wherein the labeling with specific and non-specific probes occurs simultaneously or concurrently (in either order), (3) analyzing individual nucleic acid fragments for the presence of signal from the specific and non-specific probes, and (4) obtaining, for fragments having signal from the specific probes, a profile of signals from the specific and non-specific probes along the length of the fragment.

In another aspect, the invention provides a method comprising (1) labeling nucleic acid fragments with a non-specific nucleic acid probe, such as a bisPNA probe, (2) analyzing individual nucleic acid fragments for the presence of signal from non-specific probe such as a bisPNA probe, and (3) obtaining, for fragments having signal from the non-specific probe such as a bisPNA probe, a profile of signals from the non-specific probe such as a bisPNA probe, and optionally including signals from more than one non-specific probe (if the nucleic acid was so labeled) along the length of the fragment.

Thus, in yet other aspects, the invention encompasses labeling a nucleic acid fragment with one or more non-specific probes, or one or more specific probes, obtaining signals from the non-specific probes and/or the specific probes, and separating nucleic acids based on the presence of signals from non-specific probes and/or specific-probes. Signals can be obtained and nucleic acids can be separated from each other using a microfluidic device. The probes may be bisPNA probes and the entire method may be carried out on the basis of signals from such probes.

More than one non-specific probe (e.g., bisPNA probe) can be used with the same label. More than one non-specific probes (e.g., bisPNA probe) can be used with different labels. Such probes, whether identically or differentially labeled, may differ in their binding specificity. As an example, one might bind to AT-rich regions and one may bind to GC-rich regions.

The non-specific probes in the context of aspects of invention are probes complementary to short nucleic acid sequences (e.g., 6-8 bases) that are present in genomes on average about once every 10 kb. Thus, the sequences are relatively frequent in a genome. In contrast, the specific probes of aspects of the invention are probes that are specific to sequences that exist uniquely in target genes (and thus can be used as a marker of a target gene). The profile of signals from the specific and/or the non-specific probes along the length of the fragment may be referred to herein as a signature.

It is to be understood that in addition to the foregoing probes, various aspects and embodiments of the invention also contemplate labeling of nucleic acids with backbone stains. Such stains bind along the entire length of the nucleic acid in a sequence-independent (or sequence non-specific) manner. These stains are to be contrasted with the specific or non-specific probes that bind to nucleic acids in a sequence-dependent manner (i.e., their binding to a nucleic acid is dependent upon its underlying nucleotide sequence).

In some embodiments, the method further comprises separating fragments having signal from the specific probes from fragments lacking signal from specific probes. In some embodiments, the method comprises separating fragments having certain signals (or patterns) from the non-specific probes from fragments lacking such signals (or patterns). This allows the analysis to continue on only those fragments of interest (e.g., those having signal from the specific probes). In some instances, the nucleic acid fragments of interest, whether they be identified on the basis of specific or non-specific probes or a combination of specific and non-specific probes, are rare in the population of nucleic acids.

In important embodiments, the separation of fragments occurs in a microfluidic device. In some embodiments, all or any or any combination of steps (2) through (4) and the separation step occur in the same microfluidic device. The nucleic acids may be present in the same fluid that are used as sheathing fluids, or at a minimum they are present in fluids that are miscible with the sheathing fluids. The nucleic acids are not encapsulated in droplets.

In some embodiments, the method further comprises sequencing fragments, such as fragments having signal from specific probes or fragments having signal from non-specific probes such as bisPNA probes.

In some embodiments, the method further comprises fragmenting genomic DNA to produce the nucleic acid fragments. In some embodiments, the nucleic acid fragments are generated using restriction enzyme digestion. The restriction enzyme digestion may involve the use of one or more restriction enzymes. The fragments may be of various lengths, including for example 10-500 kb (kilobases), 30-500 kb, 50-500 kb, 50-400 kb, 50-300 kb, 50-200 kb, or 50-100 kb. The fragments may be about or more than 100 kb, about or more than 200 kb, about or more than 300 kb, about or more than 400 kb, or about or more than 500 kb.

In some embodiments, the specific and/or non-specific probes are labeled with fluorophores. In some embodiments, the specific probes are labeled with multiple detectable labels and the non-specific probes are labeled with fewer detectable labels.

In some embodiments, the nucleic acid fragments are labeled under non-denaturing conditions.

In another aspect, the invention provides a method comprising (1) labeling nucleic acid fragments with three nucleic acid probes, wherein two of the probes are bisPNA probes and one of the probes is a non-bisPNA probe that is distinguishable from the bisPNA probes based on for example different brightness or intensity (e.g., it may be labeled with two or more detectable labels or with detectable labels of greater brightness or intensity while the bisPNA probes may be labeled with fewer detectable labels or with detectable labels of comparatively lesser brightness or intensity), (2) labeling the fragments with a non-specific probe, wherein the non-bisPNA probe and the non-specific probe are labeled with different detectable labels, and wherein the labeling steps of (1) and (2) may occur simultaneously or concurrently (in either order), (3) analyzing individual nucleic acid fragments for the presence of signal from the non-bisPNA and non-specific probes, and (4) obtaining, for fragments having signal from the non-bisPNA probe, a profile of signals from the non-bisPNA and non-specific probes along the length of the fragment.

In this and other embodiments, detectable labeling that is distinguishable based on intensity or brightness may be achieved using for example varieties of fluorophore-impregnated beads, quantum dots, dendrimeric probes, and the like.

In some embodiments, the method further comprises separating fragments having signal from the non-bisPNA probes from fragments lacking signal from non-bisPNA probes. In some embodiments, the separating occurs in a microfluidic device such as a microfluidic chip. In some embodiments, all or any, or some combination of steps (2) through (4) and the separating step occur in a microfluidic device. In some embodiments, the method further comprises sequencing fragments having signal from non-bisPNA probes.

In another aspect, the invention provides a method comprising (1) nicking nucleic acid fragments with an engineered restriction enzyme that possesses nicking activity in only one domain, extending a 3′ end at a nicked site using a polymerase that lacks 5′->3′ exonuclease activity to generate a displaced single strand, and labeling the displaced single strand with a specific probe that comprises at least two detectable labels or is otherwise detectably labeled in a manner that distinguishes it from other probes based on increased intensity or brightness, (2) labeling the fragments with a non-specific probe, wherein the specific probe and the non-specific probe are labeled with different detectable labels, (3) analyzing individual nucleic acid fragments for the presence of signal from the specific and non-specific probes, and (4) obtaining, for fragments having signal from the specific probe, a profile of signals from the specific and non-specific probes along the length of the fragment.

In some embodiments, the method further comprises separating fragments having signal from the specific probes from fragments lacking signal from the specific probes. In some embodiments, the separating occurs in a microfluidic device such as a microfluidic chip. In some embodiments, all or any or any combination of steps (2) through (4) and the separating step occur in a microfluidic device. In some embodiments, the method further comprises sequencing fragments having signal from specific probes.

In another aspect, the invention provides a microfluidic device comprising an inlet port coupled to a microfluidic taper comprising an elongation region and interrogation region, a cathode and anode positioned on opposite ends (i.e., of an axis) of a sorting channel that is present at or near the end of the microfluidic taper and that is oriented perpendicular to the microfluidic channel, a first DNA reservoir coupled to the sorting channel, optionally having a second elongation region and a second interrogation region located between the sorting channel and the first DNA reservoir, a second DNA reservoir positioned downstream of the sorting channel, optionally coupled to a third elongation region and a third interrogation region, and an outlet waste port downstream of the microfluidic taper, sorting channel, and second DNA reservoir, wherein a vacuum may be applied to the first and second DNA reservoirs, and the waste port.

In another aspect, the invention provides a microfluidic device comprising an inlet port coupled to a microfluidic taper comprising an elongation region and interrogation region, a cathode and anode positioned on opposite ends of a sorting channel that is present at or near the end of the microfluidic taper. The sorting channel is located beyond (or after) the microfluidic taper. In some embodiments, the device comprises two exit ports, one for discarded nucleic acids and one for selected nucleic acids. The stretching module may be separated from the sorting module by a distance that is sufficiently long to provide any delay required to perform analysis of the nucleic acid and comparison with templates in a database. In one embodiments, electrodes generate fields that are non-parallel to the direction of nucleic acid movement (and which may be perpendicular or at an angle to the direction of nucleic acid movement.

In yet other aspect, the invention provides a microfluidic device comprising an inlet port coupled to a microfluidic taper comprising an elongation region and interrogation region, a reservoir beyond the interrogation region where nucleic acids are sheathed by side fluid flows, and two exit ports, one each for discarded and selected nucleic acids. Discarded and selected nucleic acids can be directed to the corresponding ports by changing the balance of sheathing flows. In both cases, nucleic acids and sheathing flows can be driven either by positive pressure applied to the inlet and sheathing ports, or by vacuum applied to the exit ports, or by a combination thereof. The flows can be also controlled by positive pressure applied to the inlet port and the sheathing fluid ports.

### BRIEF DESCRIPTION OF FIGURES

FIG. 1. Positions (base numbers) of the conservative genomic sequences in the gene of 16S rRNA used for hybridization of forward (F) and reverse (R) primers.

FIG. 2. Schematic of Genome Sequence Scanning™ (GSS™). (I) Genomic DNA is extracted from bacteria, specifically cut with a restriction endonuclease, and tagged with a fluorescent probe. (II) Tagged DNA fragments are stretched into linear conformation by microfluidics. (III) Fluorescence pattern of the hybridized probes measured for every DNA molecule. (IV) The detected patterns are compared with the database for identification.

FIG. 3. Multiple fluorescent traces generated from E. coli K12 genome by digestion with SanD1 enzyme and probes, recognizing GAGAAAGA (green) and GAAGAGAA (red) motifs. Positions of the corresponding DNA fragments are shown in the circular E. coli genome (center).

FIGS. 4A-4B. Scheme of the DNA sorting.

FIG. 4A. General sorting approach based exclusively on GSS™ analysis using non-specific probes. If the pattern (comprised of one or more signals) does not match a template, the DNA molecule proceeds to drain (t₁). Only DNA fragments with matching patterns are directed to the selected nucleic acids reservoir (t₂).

FIG. 4B. Selection using specific probes. If only probes with short cognate sequences (non-specific probes) are detected (single trace), the DNA fragment is permitted to follow the default route to drain (t₁). If the DNA fragment with a target (e.g., a housekeeping) gene is detected by the presence of specific probes (spiked intensity profile), the DNA fragment is redirected to the collection reservoir for selected fragments (t₂).

FIG. 5. Formation of a PD-loop. A DNA fragment (A) is hybridized to two bisPNA probes (B and C). If these probes are sufficiently close to each other, they displace a long strand of DNA, which in turn can be hybridized with an oligonucleotide (D), carrying two fluorophores. The whole process does not require denaturing conditions at any stage.

FIG. 6. Suitable site for PD-loop formation at position 1215 bp in the 16S rRNA gene. One of the sites for bisPNA hybridization includes a pyrimidine; therefore, bisPNA will hybridize only with 7 bases. The sites are separated by a single base.

FIG. 7. Suitable site for PD-loop formation at position 1531 bp in the 16S rRNA gene. Each of the sites for bisPNA hybridization includes a couple of pyrimidine inserts; therefore, bisPNAs will hybridize only with 7 bases, The sites are separated by 2 bases.

FIGS. 8A-8C. Single strand flap hybridization.

FIG. 8A. One strand nick is performed by a specific nicking enzyme.

FIG. 8B. The nicked DNA strand is displaced by synthesizing the complementary sequence from the nick using Vent(exo-) polymerase.

FIG. 8C. A complementary fluorescent oligonucleotide is hybridized to the displaced DNA strand.

FIG. 9. Microfluidic chip for DNA sorting. Pictures of pre-sorting DNA reader I and DNA sorting structure II are presented in panels B and C, respectively.

FIGS. 10A-10B. Scheme of a microfluidic chip for DNA sorting using hydrodynamics.

FIG. 10A shows normal flow of DNA (referred to herein as DNA, DNA fragments or DNA molecules, interchangeably) through the chip to the waste (labeled as W).

FIG. 10B shows the change of flow of selected DNA to the collection reservoir (labeled as C).

### DETAILED DESCRIPTION OF INVENTION

The invention provides, inter alia, methods and devices for rapid analysis of nucleic acid samples, including but not limited to genomic nucleic acid samples regardless of source. The methods and devices provided herein facilitate analysis, including sequencing of nucleic acid samples by directing analysis and sequencing resources towards nucleic acids and nucleic acid fragments of interest, rather than analyzing and sequencing nucleic acids and fragments that are not of interest. In some instances, those of interest may be in the minority.

With the advent of new generation sequencing (NGS) technologies, the spectrum of applications based on sequencing has progressively widened in view of the increased throughput and decreased cost. However, even with such high throughput (measured currently in tens of gigabases per run), NGS-based applications can still benefit from limiting analysis to only relevant DNA fragments. This approach is referred to as targeted resequencing. Applications that utilize or would benefit from targeted resequencing are described below.

Targeted resequencing simplifies functional analysis of genomes. Sequencing de novo of a complete genome is still far from routine, especially for the NGS technologies, most of which have short read lengths. Short reads are difficult to combine into a contiguous genome because of common or nearly common elements that are longer than a single read length (e.g., repeats or rrn operons). Analyzing genomic regions that are 5′ and 3′ of these common or near common elements requires reads that are longer than these elements or other techniques that may be slower, manual and expensive.

For the vast majority of applications, especially in human diagnostics, pharmacogenomics, etc., de novo sequencing is not necessary. Instead, sequencing is typically used to identify genetic differences between individuals, using a process called targeted resequencing. In this approach, only portions of a genome that include the genes of interest are sequenced (Ng et al., 2009; Shendure & Aiden, 2012; Glaser 2013). Some techniques exist to identify and isolate the genomic fragments of interest. An example of such a technique is exome sequencing. This approach focuses on analyzing coding sequences or exon regions.

Targeted resequencing simplifies analysis of microbiomes. This application is explained in greater detail below.

Microbiome Analysis.

Most of living mass in this world consists of bacteria. Bacterial populations including dozens to millions of species inhabit soil and air, food and water, surface of tables in restaurants and surface of walls in hospital wards, skins and intestines of animals, humans included. Every population is a dynamic system that responds with changes of its composition to every change of conditions. The bacteria constituting the populations may compete with each other or be symbiotically connected. These bacteria can be beneficial or pathogenic for the hosts they inhabit (Sachs et al. 2011).

Bacterial populations are fundamental to the existence of biomes that include every form of life. However, most of the studies so far have been centered on the human-oriented aspects. For example, among the properties of soil-dwelling bacteria, the ones attracting most interest are the ability of some to generate antibiotics and the mechanisms of the antibiotic resistance of the others (D'Costa et al. 2006). Another type of study is search for new functions, such as enzymes that efficiently deconstruct plant cellulosic biomass to employ them in the biofuel production (Hess et al. 2011).

Human-inhabiting microbiomes attract ever growing attention as they are directly relevant to human health and well-being. Bacterial populations inhabiting skin, nose, lungs, and guts of humans possibly influence various body processes such as formation of atherosclerotic plaque (Koren et al. 2011). Another example is formation of a complex microbiome in the airways of cystic fibrosis patients that influences the disease progress (Cox et al. 2010; Zemanick et al. 2010). Intestinal microbial community is rich and plastic (Arumugam et al. 2011; Koren et al. 2011; Walter et al. 2011). It is easily modified by external influences such as diet or antibiotic treatment (Dethlefsen et al. 2008; Goodman et al. 2011). These modifications may have immediate and dangerous effects on human health, for example, by promoting growth of pathogens such as Clostridium difficile (Bartlett 2002; Cohen et al. 2010).

Studies of microbial populations were gaining steam in the last decade, facilitated by the development of the NGS technologies and related methods. The two main methodologies are based on sequencing housekeeping genes and on shotgun sequencing. The other co-existing techniques are more laborious, or more expensive, or less accurate. For example, the most direct approach based on culturing microbiome components includes analysis of tens of thousands of colonies that must be grown both under aerobic and anaerobic conditions and still does not reproduce exactly the composition of the gut microbiota (Goodman et al. 2011). Isolated bacteria grow differently in cultures, and many cannot be grown at all. Even using gnotobiotic mice to achieve the most adequate conditions cannot achieve accurate quantitative representation (Goodman et al. 2011).

Microbiome Analysis Using Housekeeping Genes.

This approach is based on genes that code the most vital functions of bacteria cells and therefore evolve at a much slower pace than less important elements. These genes include conservative sequences that may be used to design primers for DNA synthesis capable of hybridizing to the genes of many different bacteria. Therefore, a limited set of primers can be used with a bacterial mixture. Once the sections of the housekeeping genes defined by primers are sequenced, the sequences are used to identify the bacteria species and their copy numbers to quantify the proportions of the species. The most popular housekeeping genes used in this analysis are the genes coding RNA of ribosomes (rRNA).

The housekeeping genes, coding the rRNA for the 5S, 16S, and 23S ribosome particles, are combined in rrn operon as well as the tRNA molecules required for protein synthesis (Klappenbach et al. 2001). Of these genes, the one coding 16S rRNA is used most often for the sequencing-based applications. This gene has most sequences determined for different bacteria; as of Apr. 10, 2011, the 16S rRNA database included 1,962,952 sequences.

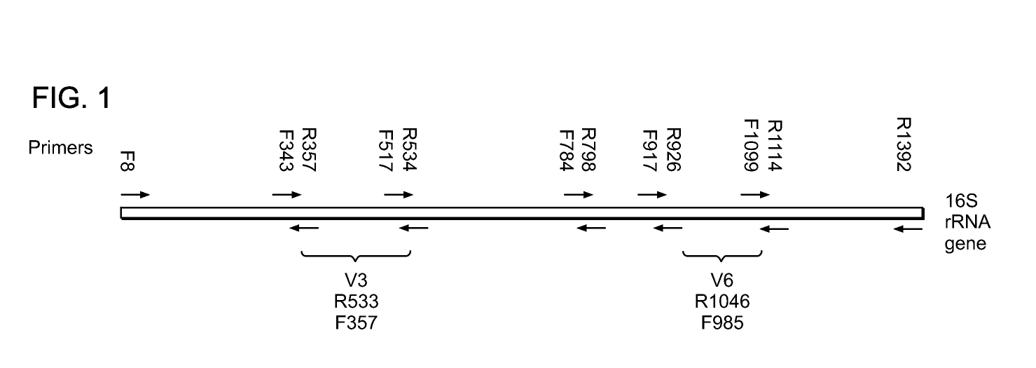

The whole 16S rRNA sequence is 1.4 kb long. It includes 7 conservative sites that are used for DNA priming (Liu et al. 2007). These sites are used to generate amplicons of various lengths (FIG. 1). The regions V3 and V6 are most often used in microbiome studies. For example, for the bacteria inhabiting human intestine the average amplicon lengths of V3 and V6 regions are 145 and 59 bp, respectively (Dethlefsen et al. 2008). The longer the length of the region used for analysis, the higher the specificity of bacteria identification that can be achieved (Liu et al. 2007; Turnbaugh et al. 2009). The whole 16S rRNA gene can be sequenced in a single run only by a classic Sanger sequencer, such as ABI 3730xl system. However, this method has the lowest throughput (192 full length 16S rRNA sequences per run) and, therefore, the highest cost of analysis. The highest throughput can be achieved with Illumina sequencing system (1.8×10⁸ reads of 100 bp length per run for GAIIx system). However, because of short read length, this system can be used for only V6 regions. Only with the recent 150PE flow cell does the Illumina system become applicable for V3 region analysis also. 454 pyrosequencing system has lower throughput than Illumina system, but higher throughput than ABI sequencer (1.4×10⁶ reads of 300-500 bp length for Genome Sequencer FLX system). To decrease costs, multiple samples can be pooled for analysis in a single run. Up to 286 samples can be pooled for a 454 sequencing system (Hamady et al. 2008). Even more samples can be combined for an Illumina system analysis.

Selection of the system for microbiome analysis is a compromise between specificity and sensitivity of detection of bacteria, because the sequencing systems with higher throughput have shorter read lengths. The longer the length of the genomic sequence used for analysis, the higher the specificity in bacteria identification that can be achieved (Turnbaugh et al. 2009). The larger the number of reads per sample, the lower the proportion of bacteria that can be detected.

A bacterium identified by a sequence-based method is referred to as an operational taxonomic unit (OTU), because the gene sequence-based recognition of uncultivated microbial populations is not equivalent to traditional taxonomic classification (Dethlefsen et al. 2008). Therefore, strictly speaking terms such as “species” or “strain” are not appropriate. OTUs can be defined in various ways and at different levels of resolution.

Full-length 16S rRNA sequences offer the highest possible degree of taxonomic resolution using this gene, but the cost of dideoxy Sanger sequencing is high (Dethlefsen et al. 2008). Less phylogenetic information is available and therefore less specific identification of bacteria is possible from a single pyrosequencing read typically covering 150-230 bp (Dethlefsen et al. 2008). In a human gut microbiome sample, for example, on average 53% of the reads could be assigned to a genus and 80% to a phylum (Arumugam et al. 2011). This relatively low specificity is only partially due to the short reads. Another problem is insufficient information content of the sequence database because abundant, uncharacterized bacterial taxa, novel species even at the family level, are still found in the human gut (Dethlefsen et al. 2008). Environmental microbiomes are even more diverse and less studied (Walter et al. 2011). In this case, the advantage of highly conservative genes is a higher chance to exhibit similarities to sequences available in a database. If higher specificity in bacterial identification is required, other markers, such as cpn60, can be used for studies (Hill et al. 2010).

Both the clone library and current NGS technologies include PCR amplification as a standard step of the sample preparation process (Metzker 2010); therefore, every NGS technology may be prone to the PCR-related artifacts, such as biased amplification. This problem is reduced for the shorter sequences required for the NGS technologies (Dethlefsen et al. 2008). With careful selection of primers no erroneous data has been generated either for the V6 or V3 variable regions or even for full-length 16S rRNA sequences (Dethlefsen et al. 2008).

Microbiome Analysis Using Shotgun Sequencing.

In this approach, total genomic DNA of the sample is isolated and fragmented, every short fragment is sequenced, and then the short reads are connected using overlaps (Hess et al. 2011; Qin et al. 2010). The assembled contigs can be used, for example, for analysis of a bacterial population by comparison with available databases of genes (Qin et al. 2010) or for mining the microbiomes for the genes coding enzymes with new functions (Hess et al. 2011). Typically, hundreds of Gigabases of sequences are obtained in these studies, several percents of which are possible to connect into contigs with a median length of 0.9-1.6 kb.

The power of this approach is that it does not require preliminary assumptions and generates information about the gene pool and enzymatic functions of the studied bacterial populations. The weakness of this approach is that it provides very limited information about the bacteria composing the microbiome. However, a paradigm presumes that the collection of the enzymes is more important for a microbiome than the particular composition of bacteria carrying these enzymes (Qin et al. 2010). Analysis of microbiomes using shotgun sequencing requires generating massive amount of data. Although the Illumina GAIIx system generates 18 Gb data per run, which is on par with all the known bacterial genome sequences combined, dozens of its runs are required to generate data for a single shotgun genomic study. And these data, once generated, require extensive computer power to generate contigs.

The invention provides in part a novel approach for carrying out targeted resequencing including analysis of microbiomes or other bulk nucleic acid populations. The approach of various aspects and embodiments of the invention utilizes Genome Sequence Scanning™ (or GSS™) which is described in greater detail herein. This approach is more general than existing approaches in the sense that one can select the subset of nucleic acid fragments to analyze further based on virtually any genetic marker or element. In addition, this approach is also automated, making for less operator error.

More specifically, in various methods of the invention, nucleic acids such as but not limited to genomic DNA may be prepared by standard methods and then may be analyzed using GSS™. Tagged DNA fragments (e.g., DNA fragments having one or more sequence-specific probes hybridized thereto) are analyzed by algorithms such as those described in published US patent application US 20120283955, the entire contents of which are incorporated by reference herein.

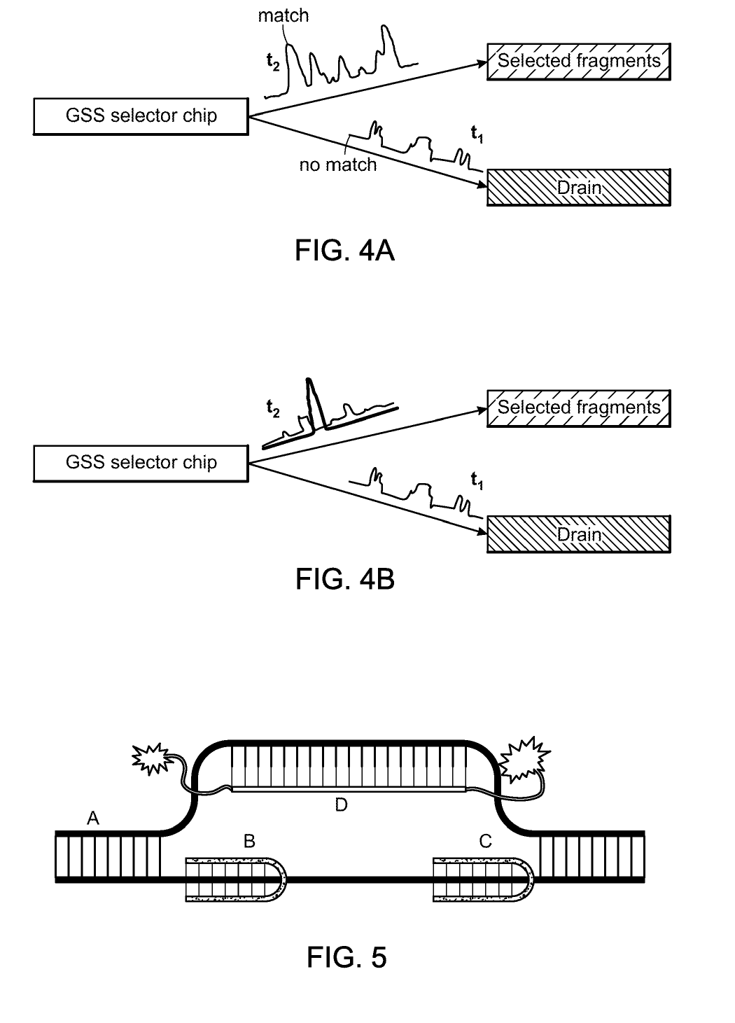

An example of such an algorithm is the “molecular classifier” algorithm (Meltzer et al., 2011). This algorithm compares profiles of single nucleic acids, such as DNA fragments, to a database of profiles. In some instances, the algorithm compares fluorescence intensity profiles generated by non-specific probes. If the desired signature is detected, the DNA fragment corresponding to that signature is re-directed (also referred to herein as “sorted”) using a selector chip or a selector module. FIG. 4A provides a schematic of such operation performed by a chip or module. It will be clear that the acquisition of the profile and its analysis must be performed while the DNA fragment is still in transit in the microfluidic chip. If the analysis is too slow, the DNA fragment will exit the chip without being redirected (sorted). Redirected DNA fragments are thus captured and available for further on-chip or off-chip analysis, including targeted resequencing. If need be, the length of the chip may be extended or velocity of the DNA can be decreased before sorting.

It is to be understood that the signatures can be used to identify DNA fragments of interest. Additionally or alternatively, they can be used for genome assembly, if desired.

Alternatively, certain short sequences may be specifically targeted (FIG. 4B). These shorter sequences may belong to conservative regions of the genome such as but not limited to housekeeping genes. An example of such a sequence and its use to select fragments is described in greater detail herein. It is to be understood that various aspects of the invention contemplate nucleic acid fragment selection based on virtually any detectable sequence, region or element, and that the examples provided herein are intended for illustration of the invention.

Various aspects of the invention further contemplate that sorting of nucleic acids can be performed using any number of detectable sequences, regions or elements provided that they are distinguishable from each other.

Genome Sequence Scanning™ (GSS™) technology, a.k.a. Direct Linear Analysis (DLA) technology, is a proprietary technology developed by Pathogenetix (PGX, formerly U.S. Genomics) for large-scale analysis of long DNA molecules and ideally suits analysis of bacterial genomes (Chan et al. 2004; Protozanova et al. 2010). The technology has been applied to detection of biopathogens in aerosols, analysis of clinical isolates of S. aureus, and speciation and typing of multiple strains of various pathogens using a single reagent set.

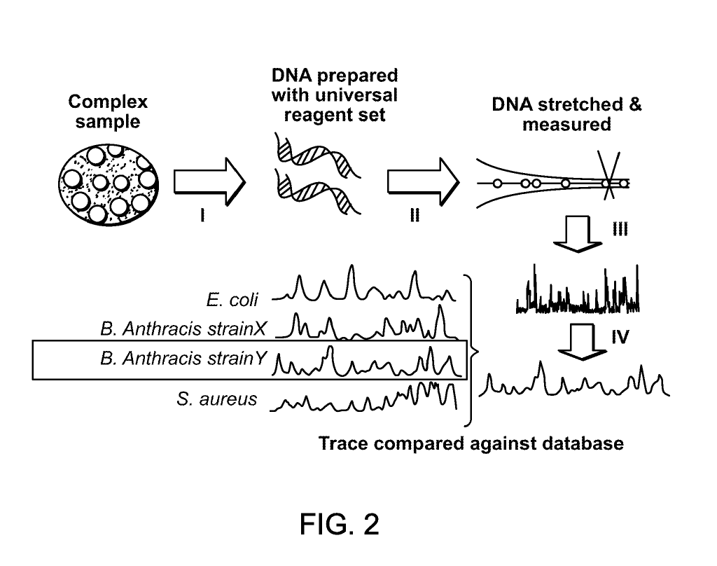

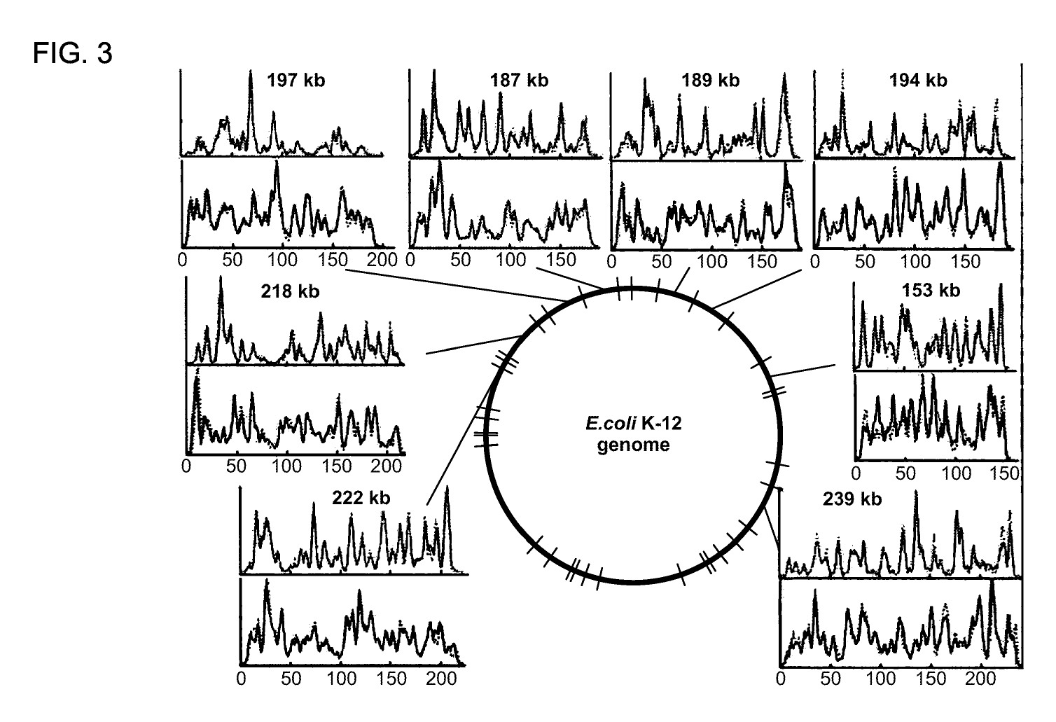

GSS™ includes isolation of genomic DNA from a bacterial sample, specific digestion with a restriction endonuclease (RE), tagging the DNA with a fluorescent sequence-specific probe, measuring the distribution pattern of probes along the DNA fragments (a signature, essentially a map of probes), and then comparing the signatures against a database for identification (FIG. 2) (Protozanova et al. 2010). GSS™ analyzes long DNA fragments (100-300 kb) and the probes hybridize to short (6-8 bases) sequences; therefore, every DNA fragment carries dozens of the probes, the positions of which depend on unique underlying genomic sequence. Typically, RE digestions generate several long DNA fragments per every genome. The signatures are information-rich and highly specific. The information content is further increased by the simultaneous use of two different-colored probes recognizing different motifs, therefore generating two independent barcodes for every fragment (FIG. 3).

Because the probes hybridize to short sequences, they bind with high probability to any genomic DNA. Therefore, unlike approaches that require specific reagents for detection of each pathogen, GSS™ employs a single reagent set to create the barcodes which can then be used to detect and identify thousands of strains from hundreds of species. The fragments of genomic DNA and the signatures generated depend on the selection of the probes and of the restriction enzyme; we call the combination of the probes and of the enzyme the signal-generating (SG) set. The RE is selected to produce the maximal number of fragments of genomic DNA in the length range between 80 and 350 kb. The probes are selected to maximize specificity of the barcodes, which typically requires high density of cognate sites. The same GSS™ instrument can be used with different SG sets, which can be optimized for different applications. SG sets for typing are finely tuned to maximize sensitivity to small genomic differences within a group of similar genomes. SG sets for speciation cover wide range of different genomes. And SG sets for detection of targets in presence of biobackground are biased to produce longer fragments of genomic DNA. However, although optimization improves GSS™ performance, even not optimized SG sets perform well and combine the ability to identify many species with high typing discrimination.

The databases used for identification can be generated either in silico from sequenced genomes or measured using cultured isolates of any bacterium. Only organisms with templates in the database can be identified. However, signatures of all microorganisms in a sample can be measured; therefore, detection of the presence of unknown microbes is possible. Also different strains of a species in the database generally carry sufficient information overlap with a newly detected strain and it can therefore its species origin or source can be determined even if the particular profile is not specifically included in the database.

GSS™ technology has potential for microbiome analysis. Because it employs the same reagent set for multiple bacteria and detects any single DNA fragment from the sample, any bacterial mixture can be processed and measured using GSS™. GSS™ can be used to detect certain target microorganisms in the bacterial mixture. Only closely related microorganisms, such as Escherichia coli and certain Shigella species, demonstrate detectable relatedness because of their genomic similarity. Otherwise, only strains of the same species exhibit sufficient resemblance for identification in the absence of exact matches in the database.

Because of the lack of signature similarity between different species, applicability of GSS™ to microbiome analysis requires presence in the database of at least some strain of the species detected in a sample. However, microbiomes in general include poorly studied bacteria, which usually have no sequenced relatives at strain level (Dethlefsen et al. 2008; Walter et al. 2011). Populating a database by direct measurements of reference samples is also impractical, because even similar microbiomes, such as guts of different humans, include very different bacterial populations (Arumugam et al. 2011; Dethlefsen et al. 2008).

Some aspects of the invention permit combining the generality of analysis based on sequencing of either complete housekeeping genes or their portions with the specificity of GSS™ that can identify bacteria down to species or even strain level.

In one aspect, the invention provides a method that comprises
(1) providing a population of nucleic acid fragments,
(2) labeling the fragments with a nucleic acid probe that is specific for a housekeeping gene or other genetically conserved sequence (i.e., the specific probe),
(3) labeling the fragments with a nucleic acid probe that is a non-specific probe (i.e., the non-specific probe), wherein the specific probe and the non-specific probe are labeled with different detectable labels,
(4) analyzing individual nucleic acid fragments for the presence of signal from the specific and non-specific probes,
(5) obtaining, for fragments having signal from the specific probes, a profile of signals from the specific and non-specific probes along the length of the fragment,
(6) separating fragments having signal from the specific probes from fragments lacking signal from specific probes, and optionally
(7) sequencing fragments having signal from non-specific probes.

The profile obtained in step (5) can be used to identify the source of the fragment (e.g., the species and/or strain of bacteria). In some embodiments, the source of the fragment is identified by sequencing nucleic acid regions adjacent to the binding site of the specific probe.

In some embodiments, the specific and non-specific probes are labeled with the same detectable label and are the signals from these are distinguished from each other based on intensity.

In some embodiments, the method comprises steps (1) through (4), or steps (1) through (5), or steps (1) through (6), or steps (1) through (7). In some embodiments, the method comprises steps (1) through (4) and (6) and optionally (7) also.

In some embodiments, the method further comprises fragmenting a population of nucleic acids in order to form the population of nucleic acid fragments. The population of nucleic acids will typically be obtained from a cell population such as a cell population from a biological sample (e.g., a gut sample, a lung sample, a stool sample, and the like).

As used herein, labeling fragments with a probe means that the fragments are exposed to the probe and, if the fragment comprises the probe's binding site, then the probe binds to the fragment. The probe's binding site may be a sequence complementary to the probe's sequence. Although shown above as two separate steps, labeling of the fragments with the specific and non-specific probes may occur simultaneously.

As used herein, fragments having signal from a probe means fragments having probes bound to them.

As used herein, a profile of signals from the probes along the length of the fragment means an intensity versus length (or distance) plot for an individual nucleic acid fragment. Examples of such a profile are provided in FIG. 4.

As used herein, a probe that is labeled with a detectable label is a probe that is conjugated to a detectable label. The detectable label may be a fluorophore but it is not so limited.

Detectable labels include fluorophores such as organic fluorophores (e.g., TMR or fluorescein), polystyrene beads impregnated with fluorophores such as organic fluorophores, dendrimers carrying conjugated fluorophores, and the like. Detectable labels also include inorganic fluorescent moieties such as quantum dots.

Various embodiments are described in greater detail below.

Specific Probes:

Specific probes are typically specific for conserved gene or nucleic acid regions. An example of a conserved gene is a housekeeping gene. In some embodiments, the housekeeping genes belong to the rrn operon. In some embodiments, the housekeeping gene encodes 16S rRNA.

The specific probe should be designed such that it binds only to its target. Typically, this means that it will be sufficiently long and therefore will only find complementarity to its target and not to other nucleic acid regions that by chance have the same sequence. As will be discussed below, this characteristic is in contrast to the non-specific probe used in certain methods described herein. There may be multiple copies of the housekeeping gene of interest. As a result, fragments may contain one or more binding sites for the specific probe and/or a genome may contribute one or more fragments having a specific probe binding site to the population of fragments. Finally, housekeeping genes tend to be conserved across species and strains of species and therefore it is possible to analyze a number of species and strains using the same probes. This facilitates the analysis.

The specific probes are labeled with a detectable label. The detectable label may be a fluorophore. The detectable label on the specific probe typically is different from the detectable label on the non-specific probe. For example, the specific probe may be labeled with a fluorophore emitting in the red wavelength range and the non-specific probe may be labeled with a fluorophore emitting in the green wavelength range.

In some embodiments, the specific probes are labeled with multiple detectable labels. This results in the intensity of signal from such probes to be greater than the intensity of signal from the non-specific probes. Intensity alone may therefore be used to distinguish between a specific probe signal and a non-specific probe signal.

FIG. 4 shows profiles having signals from specific and non-specific probes. As will be apparent, the peaks corresponding to the specific probes can be distinguished from the peaks corresponding to the non-specific probes based on color, number of peaks (e.g., usually only one specific peak and multiple non-specific peaks from a single fragment), and intensity of peak (e.g., a higher intensity peak from a specific probe compared to a non-specific probe).

At least two schemes exist that achieve the goals of specific binding and high intensity signals. These are PD-loops (Demidov et al. 2001) and single flap hybridization (Das et al. 2010). In both schemes, exceptional specificity is achieved by the combination of several independent events, such as hybridization of probes or single strand nicking, within a narrow region around the tagging site. Both schemes permit introduction of at least 2 fluorophores per site.

PD-Loops:

PD-loops are formed by hybridization of two bisPNA probes in close proximity to each other on a target DNA (FIG. 5). If the binding sites of the two bis PNA probes are within 12-14 bases, they displace a DNA strand that cannot re-hybridize with its complementary DNA sequence between the probes. Therefore, two short bisPNAs displace a long DNA strand and maintain its single stranded conformation. If a non-bisPNA oligonucleotide complementary to the displaced DNA strand is added, it can hybridize to the displaced DNA strand. Although the bisPNAs are short and may have other cognate sites on a long DNA fragment, the formation of the complete PD-loop structure which requires both bisPNAs hybridized in close proximity is a very rare event and can ensure unique tagging even in very large genomes (Demidov et al. 2001). Because the whole process is performed under non-denaturing conditions, the non-bisPNA oligonucleotides can hybridize only if both bisPNAs are bound to the DNA fragment. If only the non-bisPNA oligonucleotide carries fluorophores, the fluorescent signal can be generated only if all three components are in place. Therefore, PD-loop tagging ensures extremely high specificity, far surpassing the specificity that can be achieved with any single probe.

Specificity of this method can be improved even further if the bisPNA probes have short cognate sequences (6-7 bases) and cannot form stable complexes except if both are present and in close proximity to each other (Phillips et al. 2005).

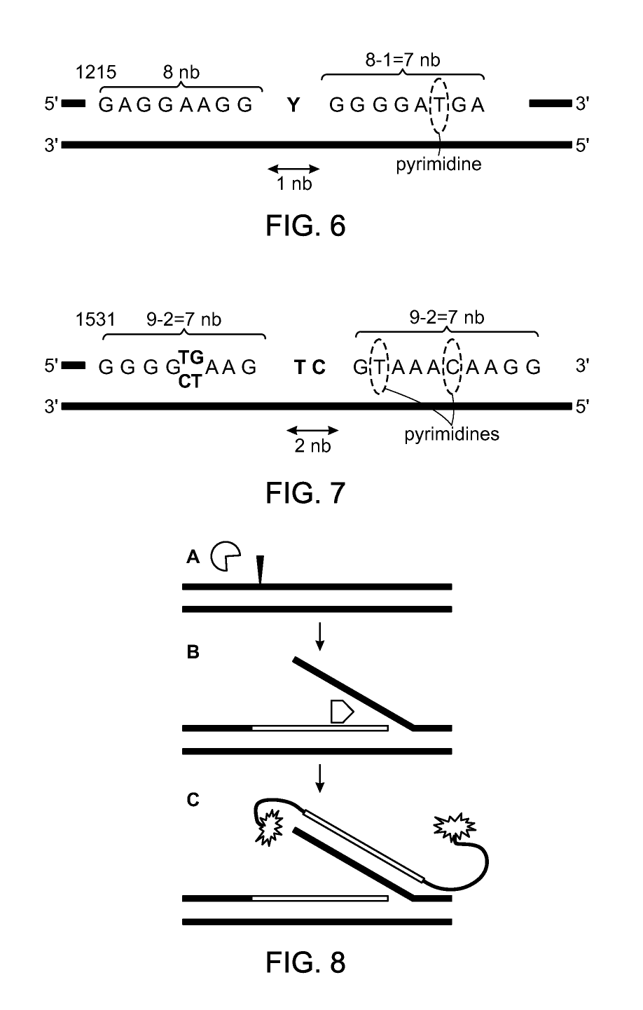

BisPNA requires a polypurine target site to hybridize. The presence of two polypurine runs of sufficient length in close proximity is relatively rare. To assess if this approach is possible, for example, with the 16S rRNA gene, we studied sequences of 10 genes in 9 bacteria: Acidaminacoccus fermentas VR4, Bacteroides fragilis YCH46, Escherichia coli K12 MG1655, Escherichia coli O157:H7 TW14359, Prevotella ruminicola 23, Ruminococcus albus 7, S. enterica Typhimurium LT2 (2 different genes), Shigella flexneri 2a str. 2457T, Staphylococcus aureus Mu3. This set includes representatives of commensal and pathogenic intestinal microflora, gram-positive and gram-negative bacteria, different and similar (E. coli and S. flexneri) species, and two strains of the same species (E. coli). Therefore this set is representative of the bacteria that can be encountered in a gut microbiome. We have identified sufficiently conservative regions that are the same in every bacterium, and searched for the sites suitable for formation of PD-loops. We found two suitable regions, starting at 1215 bp and 1531 bp (FIGS. 6 and 7, respectively) that could be used for PD-loop formation.

Single Strand Flap Hybridization:

In this method illustrated in FIG. 8, one of the DNA strands is specifically cut by a nicking enzyme (Das et al. 2010). This may be carried out using an artificial Type II restriction endonuclease, which retains the ability to bind specifically to its cognate DNA sequence while its ability to cut DNA backbone is inactivated in one of its domains (thereby resulting in nicks in the DNA rather than double-stranded cuts). After nicking, the 3′-terminus at the DNA cut is extended using a polymerase that retains displacement activity, but lacks 5′→3′ exonuclease activity (such as Vent (exo) polymerase). As a result, the DNA strand with a 5′-terminus at the DNA cut is displaced by the extended DNA chain (FIG. 8B). If an oligonucleotide complementary to the displaced strand is added, it can hybridize with the displaced DNA strand. Even though the nicking enzyme may generate nicks at some other sites, the oligonucleotide probe is not designed to be complementary to the DNA in their vicinity. Because the whole process is performed under non-denaturing conditions, the oligonucleotide can hybridize only with the single stranded flaps. The specificity of this technique is based on the combination of two requirements—the cognate site for the nicking enzyme must be in close proximity to the DNA sequence that is complementary to the oligonucleotide probe. Only the oligonucleotide probe carries detectable labels such as fluorophores; hence, only the specific complexes with all three components are detected.

To assess if this approach is possible, for example, with the 16S rRNA gene, we studied the sequences of the same set of 10 genes in 9 bacteria, described in the previous section, which is representative of the bacteria that can be encountered in gut microbiome. We have identified sufficiently conservative regions that are the same in every bacterium, and searched for the sites suitable for formation of single strand flaps. We were able to find a suitable region, starting at 351 bp that can be used with Nt.BsmAI nicking enzyme.

Nicking enzymes that can be used in various aspects of the invention include but are not limited to those commercially available and those engineered from Type II restriction enzymes.

Non-Specific Probe:

The non-specific probes are probes that bind specifically to more common sequences in the genome. Accordingly, the binding sites for these probes are not specific for any particular gene or region within the genome. The binding sites for these probes are about 8 bases in length. The probe themselves may be longer particularly if they are for example bis-PNA probes. In some instances, the binding site of the non-specific probes is chosen so that it occurs multiple times per 100 kb of genomic DNA, including for example 10-15 times per 100 kb of genomic DNA.

Nucleic Acid Fragmentation:

Genomic DNA is fragmented, typically before the labeling process. Typically, the fragmentation generates at least some fragments that are at least 50 kb or at least 100 kb in length. Preferably, some fragments range in length from about 50 kb to about 350 kb. Fragmentation may be performed in any number of ways. One example is restriction enzyme digestion in which one or more restriction enzymes may be used and they may be used simultaneously or consecutively. Other methods for DNA fragmentation, such as controlled shearing in narrow channels, can be used.

The fragmented genomic DNA is then labeled with the two types of probes described above and is subjected to standard GSS™ measurement. As a result of the measurement, the presence of every DNA fragment is detected together with the presence and positions of the bound probes of both types. Detection of the specific (housekeeping gene) probe signals the presence of the housekeeping gene and determines its position on the DNA fragment. For every DNA fragment, the positions of the multiple non-specific (short binding site) probes are determined, thereby generating the map of the cognate sites.

The detection of the specific (housekeeping gene) probe initiates a sorting impact (deflection) that redirects the DNA fragment bound to the specific probe into an alternative exit channel (FIG. 4). This way, the DNA fragments containing the housekeeping genes with the surrounding genomic DNA are separated from the bulk DNA, accumulated, and optionally used for further analysis including sequencing.

An exemplary use of the technology is the analysis of microbiomes. The selected DNA fragments include housekeeping genes (typical size of the operons <10 kb) with long lengths of adjacent genomic DNA (50-350 kb). Moreover, every DNA fragment is accompanied by the map of the second probe (i.e., the short non-specific sites). This arrangement permits further analysis such as sequencing of sections of the 16S rRNA genes using one of the standard techniques, and therefore exploits the power of detectable relatedness of the conservative sequences even between distant relatives. This analysis however typically permits identification no better than down to family or genus level. However, the genomic sequences adjacent to the conserved regions carry more detailed information that may permit identification and discrimination between bacteria according to species and strain. Importantly, by using GSS™ analysis before-hand, most of the bulk DNA is removed, which facilitates application of shotgun sequencing. The availability of bisPNA maps accompanying every DNA fragment helps the assembly of contigs, further helping shotgun sequencing. Finally, the bisPNA maps alone in combination with the housekeeping genes may be sufficient for better specificity of the analysis of bacterial populations, even without shotgun sequencing.

The technique may be used for the analysis of bacterial mixtures. It may also be used to analyze mammalian cell populations.

In a mammalian cell context, one or more housekeeping genes may be targeted in a single experiment (i.e., more than one specific probe type may be used, each specific for a particular housekeeping gene). For example, multiple housekeeping genes of a mammalian genome can be selectively tagged and separated through the proposed process, and then optionally subjected to more detailed analysis (such as sequencing) of the entire genes and their adjacent regions.

Another possible application of the technology is in cancer diagnostics. In this embodiment, the specific probes can be specific for known oncogenes or other genomic cancer markers rather than housekeeping genes. The GSS™-based sorting selects only the DNA fragments including these specific genes, eliminating the bulk of the DNA and simplifying the analysis. Simplification of the DNA material is especially important in the detection of malignancies, typically accompanied by multiple genomic distortions involving shuffling of large DNA segments, which are different for different cells even within the same tumor (Chaffer and Weinberg 2011; Hanahan and Weinberg 2011; Stratton 2011).

For analysis of the populations of mammalians with known genome sequence, the technique can be used in its simplest form. In this case, the size and probe signature of restriction fragments containing genes of interest (or target genes) can be determined in silico using the genomic sequence. No specific probe tagging is required and DNA selection can be performed solely on the basis of the signature generated by the non-specific probes.

On-Fly Sorting:

The prototype for on-chip sorting of DNA, including the chip, electronics, and software, have been developed and successfully tested. It successfully separated BAC 12M9 (185 kb) from Lambda phage DNA (48 kb) or from a Digital DNA™ octamer (80 kb) in real time.

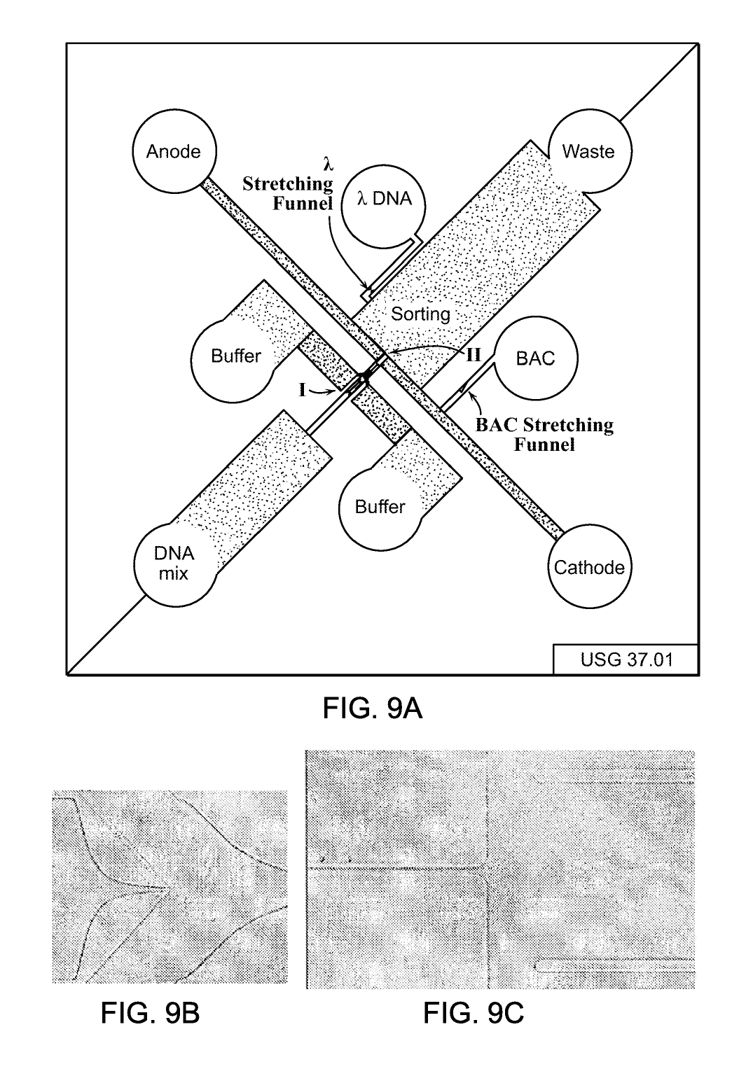

In the prototype chip, a mixture of DNA molecules of different lengths was injected into the entrance (inlet) port, marked “DNA mix” in FIG. 9A. The DNA molecules were carried hydrodynamically through the microfluidic taper labeled “I” (see also FIG. 9B), where they were stretched and their optical signals were detected. These signals were analyzed in real time. In this simple prototype, only the DNA lengths were measured; however, complete GSS™ information can be used in a similar way to distinguish between DNA fragments of the same length, but with different underlying genomic sequences. Accordingly, DNA fragments can be analyzed on the basis of binding of one or more non-specific probes, wherein the pattern of binding of the non-specific probes provides the identity of the DNA fragment.

The following is a description of a use of the chip. If a shorter DNA fragment is detected, no action is performed and the fragment proceeds further towards the port labeled “Waste” in the scheme. However, if a longer DNA molecule is detected, an electric pulse is applied between the positive and negative electrodes inserted into the ports labeled “Anode” and “Cathode,” respectively. As a result, the longer fragment is directed into the sorting zone labeled “II” (see also FIG. 9C) towards the port labeled “BAC.”

This chip was designed for GSS™ measurements with enhanced accuracy. The first microfluidic taper, preceding the sorting, was designed to stretch a wide range of molecule lengths. Its performance can be enhanced by introduction of the sheath flow delivered through the ports labeled “Buffer” (see also the microfluidic configuration in FIG. 9B). After the DNA fragments are sorted and accumulated in the different zones, vacuum driving the flow is switched from the port “Waste” to the port “λ DNA”. As a result, shorter DNA molecules are directed to the λ stretching funnel, optimized for short DNA molecules, and measured with high resolution. Then vacuum is switched from the port “λ DNA” to the port “BAC”. As a result, longer DNA molecules are directed to the BAC stretching funnel, optimized for long DNA molecules, and measured with high resolution.

For the techniques described herein, simpler chips may be used that do not require secondary stretching and rather just comprise reservoirs to accumulate the sorted fragments. Such chips have been developed and tested.

The sorting system, including the chip, flow controls, electronics, and software, can be added to the existing prototypes and future devices as an option, which does not require other modifications of the system.

It is to be understood that the sorting applications of the invention do not require the use of droplets or immiscible fluid types. Thus, the sorting applications occur without compartmentalizing DNA fragments of interest in a droplet such as an oil in water droplet or the like.

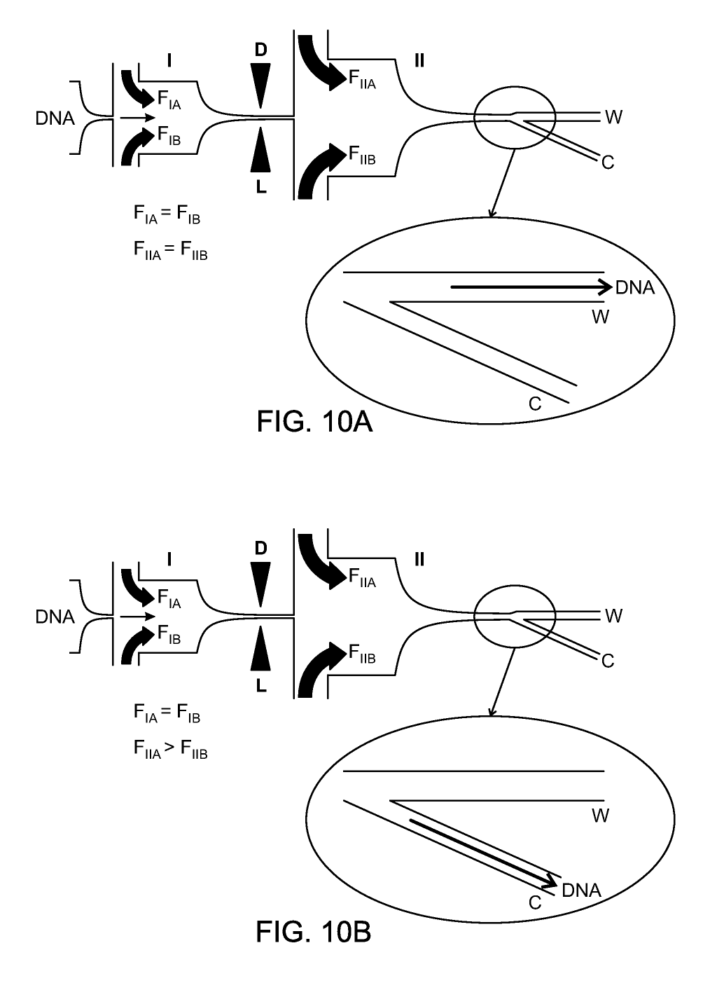

The invention contemplates in various aspects and embodiments that DNA fragments of interest may be sorted using electric or hydrodynamic forces. In electrodynamic applications, charges are applied to the stream in which the DNA fragments exist in order to direct the fragments into waste or collection reservoirs. In hydrodynamic applications, the balance of flows is distorted in order to direct the fragments into waste or collection reservoirs. This latter approach is shown in FIGS. 10A and B. FIGS. 10A and B illustrate a microfluidic chip (labeled I) that stretches probe bound DNA fragments and performs GSS™ measurements. A fluid comprising DNA is injected into the device (or system) and is immediately sheathed with two symmetric flows of a carrying buffer (labeled F_{IA} and F_{IB}). These focusing flows center the DNA in the middle of the channel and carry it through the taper, through which the flow accelerates and thereby unwinds the DNA. In their stretched conformation, DNA molecules are conveyed across the spots of laser light (labeled L), which excites fluorescence of the DNA-bound non-specific and (if any) specific probes. This fluorescence is detected in the detection module (labeled D) while the DNA passes through. As a result, the fluorescence intensity profile is detected that is characteristic of the distribution of the probes along the DNA which in turn is defined by the underlying genomic sequence of the DNA. The fluorescence intensity pattern detected for the DNA molecule is called a trace (or a profile).

While the DNA molecule continues to travel, including into and through the microfluidic subsystem II, its trace may be compared with a database that contains various templates of interest to identify the fragment of interest. The templates may be generated experimentally (and thus may be single traces or an average of multiple traces), or they may be generated non-experimentally (e.g., through prediction based on known sequence of the DNA fragment and the probes to be used). In another embodiment, the presence of specific probe is detected that identifies the fragments of interest.

In the normal state, the focusing flows of the subsystem II are also symmetric (F_{IIA}=F_{IIB}) and the path of the DNA is directed to the waste port (labeled W). This state is maintained until the system detects a DNA molecule of interest. Once a it is detected, the balance of the sheathing flows changes (F_{IIA}>F_{IIB}), and the desired DNA molecule is directed to the collection port C.

### REFERENCES

Arumugam M, Raes J, Pelletier E, Le Paslier D, Yamada T, Mende D R, Fernandes G R, Tap J, Bruls T, Batto J-M et al. 2011. Enterotypes of the human gut microbiome. Nature 473:174-180.
Bartlett J G. 2002. Antibiotic-Associated Diarrhea. New England Journal of Medicine 346:334-339.
Chaffer C L, Weinberg R A. 2011. A Perspective on Cancer Cell Metastasis. Science 331:1559-1570.
Chan E Y, Goncalves N M, Haeusler R A, Hatch A J, Larson J W, Maletta A M, Yantz G R, Carstea E D, Fuchs M, Wong G W et al. 2004. DNA mapping using microfluidic stretching and single-molecule detection of fluorescent site-specific tags. Genome Research 14:1137-1146.
Cohen S H, Gerding D N, Johnson S, Kelly C P, Loo V G, McDonald L C, Pepin J, Wilcox M H. 2010. Clinical practice guidelines for Clostridium difficile infection in adults: 2010 update by the Society for Healthcare Epidemiology of America (SHEA) and the Infectious Diseases Society of America (IDSA). Infect Control Hosp Epidemiol 31:431-55.
Cox M J, Allgaier M, Taylor B, Baek M S, Huang Y J, Daly R A, Karaoz U, Andersen G L, Brown R, Fujimura K E et al. 2010. Airway Microbiota and Pathogen Abundance in Age-Stratified Cystic Fibrosis Patients. PLoS ONE 5:e11044.
Das S K, Austin M D, Akana M C, Deshpande P, Cao H, Xiao M. 2010. Single molecule linear analysis of DNA in nano-channel labeled with sequence specific fluorescent probes. Nucleic Acids Research 38:e177.
D'Costa V M, McGrann K M, Hughes D W, Wright G D. 2006. Sampling the antibiotic resistome. Science 311:374-7.
Demidov V V, Kuhn H, Lavrentieva-Smolina I V, Frank-Kamenetskii M D. 2001. Peptide nucleic acid-assisted topological labeling of duplex DNA. Methods: A Companion to Methods in Enzymology 23:123-131.
Dethlefsen L, Huse S, Sogin M L, Relman D A. 2008. The Pervasive Effects of an Antibiotic on the Human Gut Microbiota, as Revealed by Deep 16S rRNA Sequencing. PLoS Biol 6:e280.
Glazer V. 2013. Resequencing still being targeted. GEN 33: 1, 22-23.
Goodman A L, Kallstrom G, Faith J J, Reyes A, Moore A, Dantas G, Gordon J I. 2011. Extensive personal human gut microbiota culture collections characterized and manipulated in gnotobiotic mice. Proceedings of the National Academy of Sciences 108:6252-6257.
Hamady M, Walker J J, Harris J K, Gold N J, Knight R. 2008. Error-correcting barcoded primers for pyrosequencing hundreds of samples in multiplex. Nature Methods 5:235-237.
Hanahan D, Weinberg R A. 2011. Hallmarks of Cancer: The Next Generation. Cell 144:646-674.
Hess M, Sczyrba A, Egan R, Kim T-W, Chokhawala H, Schroth G, Luo S, Clark D S, Chen F, Zhang T et al. 2011. Metagenomic Discovery of Biomass-Degrading Genes and Genomes from Cow Rumen. Science 331:463-467.
Hill J E, Fernando W M U, Zello G A, Tyler R T, Dahl W J, Van Kessel A G. 2010. Improvement of the Representation of Bifidobacteria in Fecal Microbiota Metagenomic Libraries by Application of the cpn60 Universal Primer Cocktail. Appl. Environ. Microbiol. 76:4550-4552.
Klappenbach J A, Saxman P R, Cole J R, Schmidt T M. 2001. rrndb: the Ribosomal RNA Operon Copy Number Database. Nucleic Acids Research 29:181-184.
Koren O, Spor A, Felin J, Fak F, Stombaugh J, Tremaroli V, Behre C J, Knight R, Fagerberg B, Ley R E et al. 2011. Human oral, gut, and plaque microbiota in patients with atherosclerosis. Proceedings of the National Academy of Sciences 108:4592-4598.
Liu Z, Lozupone C, Hamady M, Bushman F D, Knight R. 2007. Short pyrosequencing reads suffice for accurate microbial community analysis. Nucleic Acids Research 35:e120.
Meltzer R H, Krogmeier J R, Kwok L W, Allen R, Crane B, Griffis J W, Knaian L, Kojanian N, Malkin G, Nahas M K, Papkov V, Shaikh S, Vyavahare K, Zhong Q, Zhou Y, Larson J W, Gilmanshin R. 2011. A lab-on-chip for biothreat detection using single-molecule DNA mapping. Lab. Chip. 11: 863-873.
Metzker M L. 2010. Sequencing technologies—the next generation. Nature Reviews Genetics 11:31-46.
Ng S B, Turner E H, Robertson P D, Flygare S D, Bigham A W, Lee C, Shaffer T, Wong M, Bhattacharjee A, Eichler E E, Bamshad M, Nickerson D A, Shendure J. 2009. Targeted capture and massively parallel sequencing of 12 human exomes. Nature 461: 272-276.
Phillips K M, Larson J W, Yantz G R, D'Antoni C M, Gallo M V, Gillis K A, Goncalves N M, Neely L A, Gullans S R, Gilmanshin R. 2005. Application of single molecule technology to rapidly map long DNA and study the conformation of stretched DNA. Nucleic Acids Research 33:5829-5837.
Protozanova E, Zhang M, White E J, Mollova E T, Ten Broeck D, Fridrikh S V, Cameron D B, Gilmanshin R. 2010. Fast high-resolution mapping of long fragments of genomic DNA based on single-molecule detection. Analytical Biochemistry 402:83-90.
Qin J, Li R, Raes J, Arumugam M, Burgdorf K S, Manichanh C, Nielsen T, Pons N, Levenez F, Yamada T et al. 2010. A human gut microbial gene catalogue established by metagenomic sequencing. Nature 464:59-65.
Sachs J L, Skophammer R G, Regus J U. 2011. Evolutionary transitions in bacterial symbiosis. Proceedings of the National Academy of Sciences 108:10800-10807.
Shendure J & Aiden E L. 2012 Nat. Biotechnol. 30: 1084-1094.
Stratton M R. 2011. Exploring the Genomes of Cancer Cells: Progress and Promise. Science 331:1553-1558.
Turnbaugh P J, Hamady M, Yatsunenko T, Cantarel B L, Duncan A, Ley R E, Sogin M L, Jones W J, Roe B A, Affourtit J P et al. 2009. A core gut microbiome in obese and lean twins. Nature 457:480-484.
Walter J, Britton R A, Roos S. 2011. Host-microbial symbiosis in the vertebrate gastrointestinal tract and the Lactobacillus reuteri paradigm. Proceedings of the National Academy of Sciences 108:4645-4652.
Zemanick E T, Wagner B D, Sagel S D, Stevens M J, Accurso F J, Harris J K. 2010. Reliability of Quantitative Real-Time PCR for Bacterial Detection in Cystic Fibrosis Airway Specimens. PLoS ONE 5:e15101.

1. A method comprising
(1) labeling nucleic acid fragments with a nucleic acid probe that is a non-specific probe,
(2) analyzing individual nucleic acid fragments for the presence of signal from the non-specific probe,
(3) obtaining, for fragments having signal from the non-specific probe, a profile of signals from the non-specific probe along the length of the fragment, and
(4) separating fragments based on their profiles.

2. The method of claim 1, wherein the non-specific probe is two or more specific probes.

3. The method of claim 1, wherein the non-specific probe is a bis-PNA probe.

4. The method of claim 1, wherein the non-specific probe is specific for AT-rich nucleotide sequences.

5. The method of claim 1, wherein the non-specific probe is specific for GC-rich nucleotide sequences.

6. The method of claim 1, wherein the non-specific probe is two or more non-specific probes that are labeled with the same detectable label.

7. The method of claim 1, wherein the non-specific probe is two or more non-specific probes that are labeled with different detectable labels.

8. The method of claim 1, wherein the method further comprises sequencing fragments separated based on their profiles.

9. The method of claim 1, further comprising fragmenting genomic DNA to produce the nucleic acid fragments.

10. The method of claim 1, wherein the nucleic acid fragments are generated using restriction enzyme digestion.

11. The method of claim 1, wherein the specific and non-specific probes are labeled with fluorophores.

12. The method of claim 1, wherein the specific probes are labeled with multiple detectable labels and the non-specific probes are labeled with fewer detectable labels.

13. The method of claim 1, wherein the nucleic acid fragments are labeled under non-denaturing conditions.

14. The method of claim 1, wherein the method is performed on one or more microfluidic devices.

15. A microfluidic device comprising
an inlet port coupled to a microfluidic taper comprising an elongation region and interrogation region,
a cathode and anode positioned on opposite ends of a sorting channel that is present at or near the end of the microfluidic taper and that is oriented perpendicular to the microfluidic channel,
a first DNA reservoir coupled to the sorting channel,
an outlet waste port downstream of the microfluidic taper.

16. The microfluidic device of claim 15, wherein a vacuum may be applied to the first DNA reservoir and the waste port.


SEQUENCE LISTING (3 sequences)

SEQ ID NO: 1 - DNA, 21 bp, Artificial Sequence
  Synthetic polynucleotide
ggggtgaagtcgtaaacaagg

SEQ ID NO: 2 - DNA, 21 bp, Artificial Sequence
  Synthetic polynucleotide
ggggctaagtcgtaaacaagg

SEQ ID NO: 3 - DNA, 17 bp, Artificial Sequence
  Synthetic polynucleotide
gaggaaggnggggatga